(19) United States
(12) Patent Application Publication
(10) Pub. No.: US 2025/0054638 A1
(43) Pub. Date: Feb. 13, 2025

(54) METHODS, SYSTEMS, PROGRAMS AND DEVICES TO PREDICT PATIENT OUTCOME USING DIFFERENTIAL IN PUPILLARY INDEX

(71) Applicant: Neuroptics, Inc., Irvine, CA (US)

(72) Inventors: Kamran Siminou, Newport Coast, CA (US); Claudio Privitera, Albany, CA (US); Jeffrey Oliver, Cedar Park, TX (US)

(21) Appl. No.: 18/930,034

(22) Filed: Oct. 29, 2024

Related U.S. Application Data
(63) Continuation-in-part of application No. 17/496,109, filed on Oct. 7, 2021.

(51) Int. Cl. G16H 50/30; G16H 40/63

(52) U.S. Cl. CPC G16H 50/30; G16H 40/63

(57) ABSTRACT

A method of assessing a health condition of a mammalian subject includes using a pupilometer to take three or more sequential measurements over a period of time of the left eye and the right eye of the subject and determining the NPI of each eye at each measurement, wherein the subject is hospitalized as a result of stroke, TBI, acute brain injury or other neurological impairment. The method also includes displaying on a display of the pupilometer a comparison between the NPI of the left and the NPI of right eye at each of the three or more sequential measurements, determining whether the subject exhibits NPI differential persistence, and assessing the subject with having an increased risk of a bad neurological or health outcome if the patient exhibits NPI differential persistence.

### PRIORITY CLAIM

This application is a continuation-in-part of U.S. application Ser. No. 17/496,109, filed on Oct. 7, 2021, the entirety of which is incorporated herein by reference.

### BACKGROUND

The present disclosure relates to methods, systems, programs and devices or instruments for monitoring anisocoria and asymmetry of reaction to stimulus between the left and right pupil of a subject, sometimes referred to herein as a “patient” or an “individual”. The methods and systems can be implemented through automated infrared pupillometry (“AIP)” using devices and instruments such as pupillometers, which are high-tech instruments that are currently used to obtain information about a pupil's response to a stimulus. As used in this disclosure, pupillometer also means a smart phone or other hand-held electronic device (or computer) that runs an application or software that imparts pupillometry capabilities, such as identifying a pupil and recording and measuring its dynamic response to a stimulus, such as a light stimulus. Non-invasive AIP allows a standardized quick and reliable quantitative assessment of pupil function including diameter, constriction and dilation velocity and latency of pupillary light response. A pupillometer is a portable handheld device for bedside application. In some neurocritical care settings, pupillometry has successfully replaced conventional pupil testing. Examples of pupillometers are described in detail in U.S. Pat. Nos. 6,116,736, 6,260,968, 6,820,979, 7,147,327, 7,967,442, 8,534,840, 9,198,570, 10,154,783, and 10,687,702, all of which are incorporated herein by reference in their entirety. Examples of Pupillometers in commercial use include the ForeSite™ Pupillometer, the NPI®-200 Pupillometer, the PLR®-100 Pupillometer, the PLR®-3000 Pupillometer, the DP-1000 Pupillometer, DP-2000 Pupillometer, the A-1000 Pupillometer, and the A-2000 Pupillometer, all made by Neuroptics®, and all incorporated herein by reference in their entirety. These types of pupillometers can be used on human or animal subjects.

For example, the NPI®-200 Pupillometer can be used to provide an objective means of assessing the pupillary light reflex across a broad spectrum of neurological diseases. NPI®-200 can measure the reactivity of a patient's pupils and generate a pupillary index called a neurological pupil index (“NPI”). NPI of less than 3.0 indicates a slow or sluggish response of the pupil to the light stimulus emitted by the pupillometer and is indicative of a substantially impaired global neurological pupil function. NPI of 0 indicates a non-reactive, immeasurable or atypical response to the light stimulus emitted by the pupillometer. Pupillometry and Neuroptics' NPI has proven to be an objective means of assessing and trending pupillary reactivity across a broad spectrum of neurological diseases or health conditions of a patient. NPI forms a surrogate marker of global pupil function, which is independent of absolute parameters such as the pupil diameter.

One area of pupillary clinical diagnosis and monitoring that has gained increased attention over the years is detecting and monitoring differences in size and asymmetry in pupillary response to stimulus between the left and right pupils. This condition where the sizes of the left and right pupils of an individual at rest are unequal is called anisocoria. A variety of potential causes for anisocoria exist, ranging from trivial or normal variation, to life threatening conditions, such as increased intracranial pressure (ICP) and brain swelling from a head injury, uncal herniation, lesions, and aneurismal compression. In general all those neurological conditions affecting the efferent pathway of the pupil system starting from the oculomotor nuclei in the midbrain up to the pupil sphincter muscle can be a cause of anisocoria.

Asymmetry in pupillary response is when the left and right pupils respond to a stimulus, such as a light stimulus, in different ways that go beyond the definition of anisocoria. For example, the pupil size between the left and right pupils at rest may be the same or very close in size, but the two pupils may react to a stimulus very differently, such as having different minimum pupil size after constriction, percent constriction, average constriction velocity, maximum constriction velocity, dilation velocity and time to reach 75% of the initial maximum pupil size after the constriction. Like anisocoria, asymmetry in pupillary response may also be indicative of an underlying medical condition.

Because anisocoria and asymmetry in pupillary response may be symptoms in some cases of very serious and immediately life-threatening conditions, it is important for medical practitioners to have tools for easy, quick and convenient ways to accurately detect, measure, view in real-time and assess differences in pupillary size and pupillary reaction to stimulus between the right and left pupils. There are, unfortunately, very few such tools presently available. Thus, there is a need for high-tech instruments, software applications or algorithms that can be executed by pupillometers, smart phones, or computers, and systems that can perform such functions and provide objective measures of anisocoria and objective measures of the difference between dynamic responses of the left and right pupil to controlled and uniform stimuli. The methods, systems, software applications and devices of the present disclosure meet this and other such needs in the art.

### SUMMARY OF THE DISCLOSURE

In one embodiment, a method of assessing a health condition of a mammalian subject is described. The method includes recording images of a response of a left pupil of the subject to a stimulus thereby resulting in a first set of sequential images; recording images of a response of a right pupil of said subject to another stimulus, thereby resulting in a second set of sequential images; displaying on a display simultaneously the first set of images and the second set of images, wherein the two sets of images are synchronized, and wherein a center of the left pupil of each image from the first set of sequential images is aligned with a center of the right pupil from the second set of sequential images on the display; detecting anisocoria in the pupillary response of the subject by comparing the first and second set of images; and displaying on the display a pupillary index representative of the level of anisocoria detected.

In another embodiment, a method of predicting the outcome of a brain injury patient is described. The method includes recording images of a response of a left pupil of the patient to a stimulus thereby resulting in a first set of sequential images; recording images of a response of a right pupil of said patient to another stimulus, thereby resulting in a second set of sequential images; displaying on a display simultaneously the first set of images and the second set of images, wherein the two sets of images are synchronized, and wherein a center of the left pupil of each image from the first set of sequential images is aligned with a center of the right pupil from the second set of sequential images on the display; detecting anisocoria in the pupillary response of the patient by comparing the first and second set of images; and displaying on the display a pupillary index representative of the level of anisocoria detected. In one embodiment, the patient is a hospitalized acute brain injury patient.

In another embodiment, a program product is described. The program product includes a computer-readable medium and computer-executable instructions recorded on the computer-readable medium for performing a method of assessing a health condition of a mammalian subject, wherein the method comprises: recording images of a response of a left pupil of the subject to a stimulus thereby resulting in a first set of sequential images; recording images of a response of a right pupil of said subject to another stimulus, thereby resulting in a second set of sequential images; causing to be displayed on a display simultaneously the first set of images and the second set of images, wherein the two sets of images are synchronized, and wherein a center of the left pupil of each image from the first set of sequential images is aligned with a center of the right pupil from the second set of sequential images on the display; detecting anisocoria in the pupillary response of the subject by comparing the first and second sets of images; and causing to be displayed on the display a pupillary index representative of the level of anisocoria detected.

In another embodiment, a program product is described. The program product includes a computer-readable medium and computer-executable instructions recorded on the computer-readable medium for performing a method of predicting the outcome of a brain injury patient, wherein the method comprises: recording images of a response of a left pupil of the patient to a stimulus thereby resulting in a first set of sequential images; recording images of a response of a right pupil of said patient to another stimulus, thereby resulting in a second set of sequential images; causing to be displayed on a display simultaneously the first set of images and the second set of images, wherein the two sets of images are synchronized, and wherein a center of the left pupil of each image from the first set of sequential images is aligned with a center of the right pupil from the second set of sequential images on the display; detecting anisocoria in the pupillary response of the patient by comparing the first and second sets of images; and causing to be displayed on the display a pupillary index representative of the level of anisocoria detected. In one embodiment, the patient is a hospitalized acute brain injury patient.

In another embodiment, a method of assessing a health condition of a mammalian subject is described. The method includes recording images of a response of a left pupil of the subject to a stimulus (a) thereby resulting in a first set of sequential images; recording images of a response of a right pupil of said subject to a stimulus (b), thereby resulting in a second set of sequential images; generating a pupillary index for the left pupil based on the response of the left pupil to stimulus (a); generating a pupillary index for the right pupil based on the response of the right pupil to stimulus (b); and providing an output to be displayed on a display, said output indicating the difference between the pupillary index of the left pupil and the pupillary index of the right pupil and displaying said output along with the recorded images of the left and right pupil.

In another embodiment, a method of predicting the outcome of a brain injury patient is described. The method includes recording images of a response of a left pupil of the patient to a stimulus (a) thereby resulting in a first set of sequential images; recording images of a response of a right pupil of said patient to a stimulus (b), thereby resulting in a second set of sequential images; generating a pupillary index for the left pupil based on the response of the left pupil to stimulus (a); generating a pupillary index for the right pupil based on the response of the right pupil to stimulus (b); and providing an output to be displayed on a display, said output indicating the difference between the pupillary index of the left pupil and the pupillary index of the right pupil and displaying said output along with the recorded images of the left and right pupil. In one embodiment, the patient is a hospitalized acute brain injury patient.

In another embodiment, a program product is described. The program product includes a computer-readable medium and computer-executable instructions recorded on the computer-readable medium for performing a method of assessing a health condition of a mammalian subject, wherein the method comprises: recording images of a response of a left pupil of the subject to a stimulus (a) thereby resulting in a first set of sequential images; recording images of a response of a right pupil of said subject to a stimulus (b), thereby resulting in a second set of sequential images; generating a pupillary index for the left pupil based on the response of the left pupil to stimulus (a); generating a pupillary index for the right pupil based on the response of the right pupil to stimulus (b); and causing a display to display an output indicating the difference between the pupillary index of the left pupil and the pupillary index of the right pupil and displaying along with the recorded images of the left and right pupil.

In another embodiment, a program product is described. The program product includes a computer-readable medium and computer-executable instructions recorded on the computer-readable medium for performing a method of predicting the outcome of a brain injury patient, wherein the method comprises: recording images of a response of a left pupil of the patient to a stimulus (a) thereby resulting in a first set of sequential images; recording images of a response of a right pupil of said patient to a stimulus (b), thereby resulting in a second set of sequential images; generating a pupillary index for the left pupil based on the response of the left pupil to stimulus (a); generating a pupillary index for the right pupil based on the response of the right pupil to stimulus (b); and causing a display to display an output indicating the difference between the pupillary index of the left pupil and the pupillary index of the right pupil and displaying along with the recorded images of the left and right pupil. In one embodiment, the patient is a hospitalized acute brain injury patient.

In yet another embodiment, a method of assessing a health condition of a mammalian subject is described. The method includes recording images of a response of a left pupil of the subject to a stimulus (a) thereby resulting in a first set of sequential images; recording images of a response of a right pupil of said subject to a stimulus (b), thereby resulting in a second set of sequential images; generating a pupillary index for the left pupil based on the response of the left pupil to stimulus (a); generating a pupillary index for the right pupil based on the response of the right pupil to stimulus (b); and comparing the pupillary index of the left pupil to the pupillary index of the right pupil to assess the health condition of the subject.

In yet another embodiment, a method of predicting the outcome of a brain injury patient is described. The method includes recording images of a response of a left pupil of the patient to a stimulus (a) thereby resulting in a first set of sequential images; recording images of a response of a right pupil of said patient to a stimulus (b), thereby resulting in a second set of sequential images; generating a pupillary index for the left pupil based on the response of the left pupil to stimulus (a); generating a pupillary index for the right pupil based on the response of the right pupil to stimulus (b); and comparing the pupillary index of the left pupil to the pupillary index of the right pupil to predict the outcome of the patient. In one embodiment, the patient is a hospitalized acute brain injury patient. In one embodiment, the prediction is that the patient will have an unfavorable health or neurological outcome if the pupillary indexes of both left and right eyes are normal but the difference in pupillary indexes between the left and right eye exceeds a threshold value.

In another embodiment, a program product is described. The program product includes a computer-readable medium and computer-executable instructions recorded on the computer-readable medium for performing a method of assessing a health condition of a mammalian subject, wherein the method comprises: recording images of a response of a left pupil of the subject to a stimulus (a) thereby resulting in a first set of sequential images; recording images of a response of a right pupil of said subject to a stimulus (b), thereby resulting in a second set of sequential images; generating a pupillary index for the left pupil based on the response of the left pupil to stimulus (a); generating a pupillary index for the right pupil based on the response of the right pupil to stimulus (b); comparing the pupillary index of the left pupil to the pupillary index of the right pupil; and causing a display to display an output indicative of the health condition of the subject.

In another embodiment, a program product is described. The program product includes a computer-readable medium and computer-executable instructions recorded on the computer-readable medium for performing a method of predicting the outcome of a brain injury patient, wherein the method comprises: recording images of a response of a left pupil of the patient to a stimulus (a) thereby resulting in a first set of sequential images; recording images of a response of a right pupil of said patient to a stimulus (b), thereby resulting in a second set of sequential images; generating a pupillary index for the left pupil based on the response of the left pupil to stimulus (a); generating a pupillary index for the right pupil based on the response of the right pupil to stimulus (b); comparing the pupillary index of the left pupil to the pupillary index of the right pupil; and causing a display to display an output indicative of a prediction of the outcome of the patient. In one embodiment, the patient is a hospitalized acute brain injury patient.

In yet another embodiment, a method of assessing a health condition of a mammalian subject is described. The method includes recording images of a response of a left pupil of the subject to a stimulus thereby resulting in a first set of sequential images and displaying on a display a pupillary index for the left pupil in connection with the first set of sequential images; recording images of a response of a right pupil of said subject to another stimulus, thereby resulting in a second set of sequential images and displaying on a display a pupillary index for the right pupil in connection with the second set of sequential images; displaying on the display simultaneously the first set of images and the associated pupillary index for the left pupil and the second set of images and the associated pupillary index for the right pupil, wherein the two sets of images are synchronized, and wherein a center of the left pupil of each image from the first set of sequential images is aligned with a center of the right pupil from the second set of sequential images on the display; and displaying on the display a patient outcome index representing a health condition of the patient, said patient outcome index being based in part on the difference between the pupillary index of the left pupil and the pupillary index of the right pupil.

In yet another embodiment, a method of predicting the outcome of a brain injury patient is described. The method includes recording images of a response of a left pupil of the patient to a stimulus thereby resulting in a first set of sequential images and displaying on a display a pupillary index for the left pupil in connection with the first set of sequential images; recording images of a response of a right pupil of said patient to another stimulus, thereby resulting in a second set of sequential images and displaying on a display a pupillary index for the right pupil in connection with the second set of sequential images; displaying on the display simultaneously the first set of images and the associated pupillary index for the left pupil and the second set of images and the associated pupillary index for the right pupil, wherein the two sets of images are synchronized, and wherein a center of the left pupil of each image from the first set of sequential images is aligned with a center of the right pupil from the second set of sequential images on the display; and displaying on the display a patient outcome index said patient outcome index being based in part on the difference between the pupillary index of the left pupil and the pupillary index of the right pupil. In one embodiment, the patient is a hospitalized acute brain injury patient.

In another embodiment, a program product is described. The program product includes a computer-readable medium and computer-executable instructions recorded on the computer-readable medium for performing a method of assessing a health condition of a mammalian subject, wherein the method comprises: recording images of a response of a left pupil of the subject to a stimulus thereby resulting in a first set of sequential images and displaying on a display a pupillary index for the left pupil in connection with the first set of sequential images; recording images of a response of a right pupil of said subject to another stimulus, thereby resulting in a second set of sequential images and displaying on a display a pupillary index for the right pupil in connection with the second set of sequential images; displaying on the display simultaneously the first set of images and the associated pupillary index for the left pupil and the second set of images and the associated pupillary index for the right pupil, wherein the two sets of images are synchronized, and wherein a center of the left pupil of each image from the first set of sequential images is aligned with a center of the right pupil from the second set of sequential images on the display; and displaying on the display a patient outcome index representing a health condition of the patient, said patient outcome index being based in part on the difference between the pupillary index of the left pupil and the pupillary index of the right pupil.

In another embodiment, a program product is described. The program product includes a computer-readable medium and computer-executable instructions recorded on the computer-readable medium for performing a method of predicting the outcome of a brain injury patient, wherein the method comprises: recording images of a response of a left pupil of the patient to a stimulus thereby resulting in a first set of sequential images and displaying on a display a pupillary index for the left pupil in connection with the first set of sequential images; recording images of a response of a right pupil of said patient to another stimulus, thereby resulting in a second set of sequential images and displaying on a display a pupillary index for the right pupil in connection with the second set of sequential images; displaying on the display simultaneously the first set of images and the associated pupillary index for the left pupil and the second set of images and the associated pupillary index for the right pupil, wherein the two sets of images are synchronized, and wherein a center of the left pupil of each image from the first set of sequential images is aligned with a center of the right pupil of each image from the second set of sequential images on the display; and displaying on the display a patient outcome index, said patient outcome index being based in part on the difference between the pupillary index of the left pupil and the pupillary index of the right pupil. In one embodiment, the patient is a hospitalized acute brain injury patient.

In yet another embodiment, a method of assessing a health condition of a mammalian subject is provided. The method includes displaying on a display the pupillary index of a left pupil of the subject, the pupillary index of a right pupil of the subject and the difference between the pupillary index of the left pupil and the pupillary index of the right pupil, wherein the pupillary index of the left pupil is determined by recording the response of the left pupil to a stimulus and calculating a pupillary index based on said response, and the pupillary index of the right pupil is determined by recording the response of the right pupil to a stimulus and calculating a pupillary index based on the response of the right pupil; and assessing the subject with having an increased risk of a bad neurological or health outcome if the pupillary index of the left pupil and the pupillary index of the right pupil are in a normal range but the difference between the pupillary indexes is greater than a threshold differential.

In yet another embodiment, a method of predicting the outcome of a brain injury patient is provided. The method includes displaying on a display the pupillary index of a left pupil of the patient, the pupillary index of a right pupil of the patient and the difference between the pupillary index of the left pupil and the pupillary index of the right pupil, wherein the pupillary index of the left pupil is determined by recording the response of the left pupil to a stimulus and calculating a pupillary index based on said response, and the pupillary index of the right pupil is determined by recording the response of the right pupil to a stimulus and calculating a pupillary index based on the response of the right pupil; and predicting an increased risk of a bad neurological or health outcome if the pupillary index of the left pupil and the pupillary index of the right pupil are in a normal range but the difference between the pupillary indexes is greater than a threshold differential. In one embodiment, the patient is a hospitalized acute brain injury patient.

In another embodiment, a program product is described. The program product includes a computer-readable medium and computer-executable instructions recorded on the computer-readable medium for performing a method of assessing a health condition of a mammalian subject, wherein the method comprises: displaying on a display the pupillary index of a left pupil of the patient, the pupillary index of a right pupil of the patient and the difference between the pupillary index of the left pupil and the pupillary index of the right pupil, wherein the pupillary index of the left pupil is determined by recording the response of the left pupil to a stimulus and calculating a pupillary index based on said response, and the pupillary index of the right pupil is determined by recording the response of the right pupil to a stimulus and calculating a pupillary index based on the response of the right pupil; and causing to be displayed on the display an output indicative of an increased risk of a bad neurological or health outcome if the pupillary index of the left pupil and the pupillary index of the right pupil are in a normal range but the difference between the pupillary indexes is greater than a threshold differential.

In another embodiment, a program product is described. The program product includes a computer-readable medium and computer-executable instructions recorded on the computer-readable medium for performing a method of assessing a health condition of a mammalian subject, wherein the method comprises: displaying on a display the pupillary index of a left pupil of the subject, the pupillary index of a right pupil of the subject and the difference between the pupillary index of the left pupil and the pupillary index of the right pupil, wherein the pupillary index of the left pupil is determined by recording the response of the left pupil to a stimulus and calculating a pupillary index based on said response, and the pupillary index of the right pupil is determined by recording the response of the right pupil to a stimulus and calculating a pupillary index based on the response of the right pupil.

In another embodiment, a program product is described. The program product includes a computer-readable medium and computer-executable instructions recorded on the computer-readable medium for performing a method of predicting the outcome of a brain injury patient, wherein the method comprises: displaying on a display the pupillary index of a left pupil of the patient, the pupillary index of a right pupil of the patient and the difference between the pupillary index of the left pupil and the pupillary index of the right pupil, wherein the pupillary index of the left pupil is determined by recording the response of the left pupil to a stimulus and calculating a pupillary index based on said response, and the pupillary index of the right pupil is determined by recording the response of the right pupil to a stimulus and calculating a pupillary index based on the response of the right pupil. In one embodiment, the patient is a hospitalized acute brain injury patient.

In yet another embodiment, a method of assessing a health condition of a mammalian subject is described. The method includes displaying on a display the number of times a left pupil of the subject had an abnormal response to a stimulus and the number of times a right pupil of the subject had an abnormal response to the stimulus; displaying on the display the number of times a left pupil of the subject had a normal response to a stimulus and the number of times a right pupil of the subject had a normal response to the stimulus; and displaying on the display the number of times there was greater than a threshold differential between the response of the left pupil and the response of the right pupil to the stimulus.

In yet another embodiment, a method of predicting the outcome of a brain injury patient is described. The method includes displaying on a display the number of times a left pupil of the patient had an abnormal response to a stimulus and the number of times a right pupil of the patient had an abnormal response to the stimulus; displaying on the display the number of times a left pupil of the patient had a normal response to a stimulus and the number of times a right pupil of the patient had a normal response to the stimulus; and displaying on the display the number of times there was greater than a threshold differential between the response of the left pupil and the response of the right pupil to the stimulus. In one embodiment the patient is a hospitalized acute brain injury patient.

In another embodiment, a program product is described. The program product includes a computer-readable medium and computer-executable instructions recorded on the computer-readable medium for performing a method of assessing a health condition of a mammalian subject, wherein the method comprises: causing to be displayed on a display the number of times a left pupil of the subject had an abnormal response to a stimulus and the number of times a right pupil of the subject had an abnormal response to the stimulus; causing to be displayed on the display the number of times a left pupil of the subject had a normal response to a stimulus and the number of times a right pupil of the subject had a normal response to the stimulus; and causing to be displayed on the display the number of times there was greater than a threshold differential between the response of the left pupil and the response of the right pupil to the stimulus.

In another embodiment, a program product is described. The program product includes a computer-readable medium and computer-executable instructions recorded on the computer-readable medium for performing a method of predicting the outcome of a brain injury patient, wherein the method comprises: causing to be displayed on a display the number of times a left pupil of the patient had an abnormal response to a stimulus and the number of times a right pupil of the patient had an abnormal response to the stimulus; causing to be displayed on the display the number of times a left pupil of the patient had a normal response to a stimulus and the number of times a right pupil of the patient had a normal response to the stimulus; and causing to be displayed on the display the number of times there was greater than a threshold differential between the response of the left pupil and the response of the right pupil to the stimulus. In one embodiment the patient is a hospitalized acute brain injury patient.

In yet another embodiment, a pupilometer is described. The pupilometer includes: a light source for generating and emitting a light stimulus to stimulate a left pupil and a right pupil; a camera for recording dynamic responses of said pupils to light stimuli emitted by the light source; a microprocessor that generates a pupillary index for each recording of a dynamic response and associates it with either the right eye if the response is of the right eye and associates it with the left eye if the response is of the left eye; a memory that can store paired pupillary indexes; and a display that displays a summary of the pupillary indexes of each of the left and right eyes, wherein the summary includes the number of times the right eye had a normal pupillary index and how many times the left eye had a normal pupillary index, how many times the right eye had an abnormal pupillary index and how many times the left eye had an abnormal pupillary index, and how many times there was a greater than threshold differential in the paired pupillary indexes between the left and right eye.

In yet another embodiment, a pupilometer is described. The pupilometer includes: a light source for generating and emitting a light stimulus to stimulate a left pupil and a right pupil; a camera for recording a dynamic response of said pupils to the light stimulus; a memory that can store data relating to the dynamic response of each pupil to the light stimulus, wherein multiple pupillary indexes for each pupil can be stored, and differentials between the pupillary indexes of the left and right eye can be stored; a microprocessor that converts the data into a graphical representation of the dynamic response of each pupil, generates a pupillary index associated with the dynamic response of each pupil, and generates a differential in the pupillary indexes between the left and right pupil for each left and right eye measurement; and a display for simultaneously displaying the graphical representations of the dynamic responses of the left and right pupils and displaying the pupillary indexes associated with each measurement of each pupil as well as the differential in the pupillary index between the left and right pupil for each left and right eye measurement, wherein the display can display multiple pupillary indexes for each eye and multiple differentials in pupillary index between the left and right eye at the same time.

In yet another embodiment, a method of predicting the outcome of a hospitalized brain injury patient is described. The method includes receiving a first set of data representing the response of a left pupil of the patient to a stimulus (a); analyzing the first set of data and generating a pupillary index representing the response of the left pupil to the stimulus (a); displaying on a display the pupillary index of the left pupil; receiving a second set of data representing the response of the right eye of the patient to a stimulus (b); analyzing the second set of data and generating a pupillary index representing the response of the right pupil to the stimulus (b); displaying on the display the pupillary index of the right pupil; and calculating the difference between the pupillary index of the left pupil and the pupillary index of the right pupil and displaying on the display a value representing the difference between the pupillary index of the left pupil and the pupillary index of the right pupil. In one embodiment, the pupillary index of the left pupil and the pupillary index of the right pupil constitute paired pupillary index values.

In another embodiment, a program product is described. The program product includes a computer-readable medium and computer-executable instructions recorded on the computer-readable medium for performing a method of predicting the outcome of a patient, wherein the method comprises: receiving a first set of data representing the response of a left pupil of a hospitalized brain injury patient to a stimulus (a); analyzing the first set of data and generating a pupillary index representing the response of the left pupil to the stimulus (a); displaying on a display the pupillary index of the left pupil; receiving a second set of data representing the response of the right eye of a hospitalized brain injury patient to a stimulus (b); analyzing the second set of data and generating a pupillary index representing the response of the right pupil to the stimulus (b); displaying on the display the pupillary index of the right pupil; and calculating the difference between the pupillary index of the left pupil and the pupillary index of the right pupil and displaying on the display a value representing the difference between the pupillary index of the left pupil and the pupillary index of the right pupil. In one embodiment, the pupillary index of the left pupil and the pupillary index of the right pupil constitute paired pupillary index values.

In yet another embodiment, a method of assessing the health condition of a mammalian subject is provided. The method includes using a pupilometer to take paired pupillary measurements of a left and right eye of the subject; using the pupilometer to determine NPI of the left eye and display it on a display of the pupillometer; using the pupillometer to determine NPI of the right eye and display it on the display of the pupillometer; calculating a differential between NPI of the left eye and NPI of the right eye and display said NPI differential on the display of the pupillometer; and determining that the patient's condition is unfavorable if the NPIs of the left and right eyes indicate normal pupillary responses for each eye but the NPI differential meets or exceeds a given threshold value.

In yet another embodiment, a method of predicting the outcome of a hospitalized acute brain injury patient is provided. The method includes using a pupilometer to take paired pupillary measurements of a left and right eye of the patient; using the pupilometer to determine NPI of the left eye and display it on a display of the pupillometer; using the pupillometer to determine NPI of the right eye and display it on the display of the pupillometer; calculating a differential between NPI of the left eye and NPI of the right eye and display said NPI differential on the display of the pupillometer; and predicting an unfavorable health or neurological outcome if the NPIs of the left and right eyes indicate normal pupillary responses for each eye but the NPI differential meets or exceeds a given threshold value.

In another embodiment, a program product is described. The program product includes a computer-readable medium and computer-executable instructions recorded on the computer-readable medium for performing a method of predicting the outcome of a patient, wherein the method comprises: processing paired pupillary measurements of a left and right eye of a brain injury patient and calculating NPI of the left eye and NPI of the right eye; causing a display to display the NPI of the left eye and the NPI of the right eye; calculating a differential between NPI of the left eye and NPI of the right eye; displaying the NPI differential in a manner that brings attention to it if the NPI differential meets or exceeds a given threshold value even if the NPIs of the left and right eyes indicate normal pupillary responses for each eye. In one embodiment the patient is a hospitalized acute brain injury patient.

In yet another embodiment, method of assessing a health condition of a mammalian subject includes using a pupilometer to take three or more sequential measurements over a period of time of the left eye and the right eye of the subject and determining the NPI of each eye at each measurement, wherein the subject is hospitalized as a result of stroke, TBI, acute brain injury or other neurological impairment. The method also includes displaying on a display of the pupilometer a comparison between the NPI of the left and the NPI of right eye at each of the three or more sequential measurements, determining whether the subject exhibits NPI differential persistence, and assessing the subject with having an increased risk of a bad neurological or health outcome if the patient exhibits NPI differential persistence.

Other features and advantages will be apparent from the following description of the various embodiments of the disclosure, which illustrate, by way of example, the principles of the disclosed devices and methods.

### BRIEF DESCRIPTION OF THE DRAWINGS

According to common practice, the various features of the drawings may not be presented to scale. Rather, the dimensions of the various features may be arbitrarily expanded or reduced for clarity. Included in the drawings are the following figures:

FIG. 1 is an illustration of a Pupillometer having a display in accordance with one embodiment.

FIG. 2 shows a screen shot of the display of the Pupillometer depicted in FIG. 1 showing results of a measurement of a pupil of an individual in response to a visual light stimulus.

FIGS. 3A-3C depict the steps taken by the handler of the Pupillometer depicted in FIG. 1 in order to take pupillary measurements.

FIG. 4 shows a screen shot of the display of the Pupillometer depicted in FIG. 1 showing the left and right pupils of an individual aligned during video playback of pupillary response to a light stimulus.

FIG. 5 shows a second screen shot from the same video described with respect to FIG. 4.

FIGS. 6-8 show different screen shots of the display of the Pupillometer depicted in FIG. 1 showing the left and right pupils of an individual aligned during video playback of pupillary response to a light stimulus along with a graphical representation of the pupillary response.

FIG. 9 shows a different screen shot of the display of the pupillometer depicted in FIG. 1 showing NPI results and size results in graphical form of a paired measurement of a left and right pupil of an individual in response to a light stimulus, along with overall patient index.

FIG. 10 shows the screen shot of the display of the pupillometer depicted in FIG. 1 showing left and right eye data from a patient.

FIG. 11 is a bar graph depicting data from stroke and TBI patients.

FIG. 12 is a set of bar graphs depicting data from the same stroke and TBI patients depicted in FIG. 11.

FIG. 13 is a bar graph depicting first occurrence of NPI differential vs. first occurrence of abnormal NPI in the patients depicted in FIGS. 11 and 12.

### DETAIL DESCRIPTION

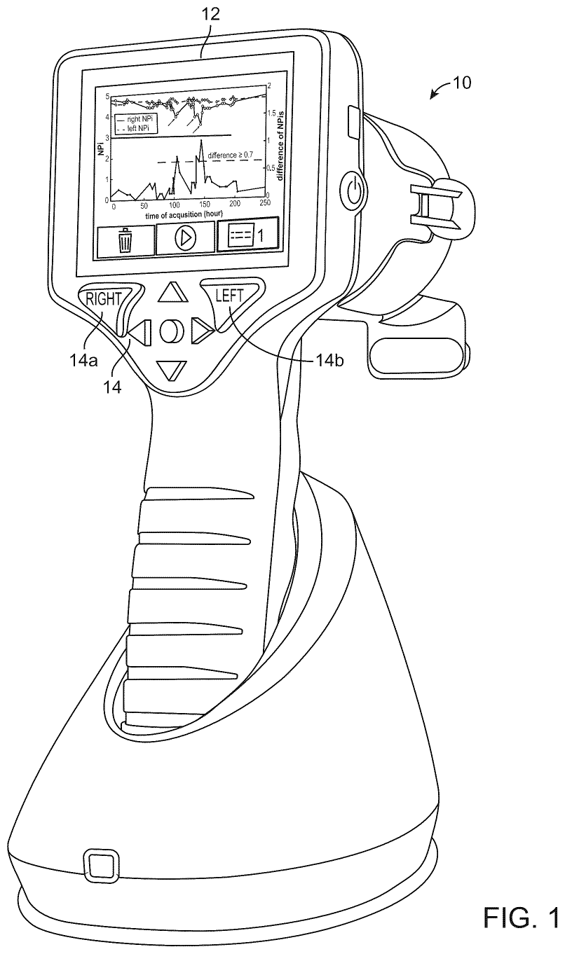

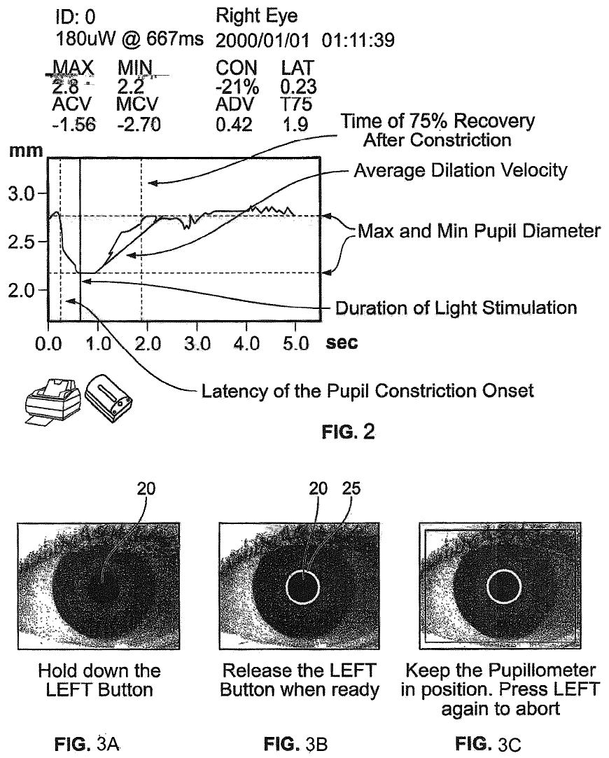

Disclosed herein is a pupillary analysis system that includes a pupillometer, such as the one shown in FIG. 1, with features that enable it to monitor and compare in real-time the response of an individual's left and right pupils to a stimulus, such as a light stimulus, auditory stimulus, or noxious stimulus. In the examples described herein the pupillometer has a light stimulus source built into it. Variations on the type of stimulus and the arrangement of that stimulus in relation to the pupillometer, such as those variations described in U.S. Pat. No. 7,147,327, are also contemplated and incorporated herein by reference. The working components, electronics, and software, other than those described below, are also fully described in, e.g., U.S. Pat. No. 7,147,327, and will therefore not be further discussed herein. As used in this disclosure, pupillometer also means a smart phone or other hand-held electronic device (or computer) that runs an application or software that imparts pupillometry capabilities, such as identifying a pupil and recording and measuring its dynamic response to a stimulus, such as a light stimulus. Thus, the various screen shots (FIGS. 2-9 herein) can be displayed on a smart phone or other hand-held electronic device (or a computer) running a pupillometry software application or program.

Before the present subject matter is further described, it is to be understood that the subject matter described herein is not limited to the particular embodiments described, and as such may of course vary. It is also to be understood that the terminology used here in is for the purpose of describing particular exemplary embodiments only, and is not intended to be limiting in any fashion, and in particular to the doctrine of equivalents. Unless defined otherwise, all technical terms used herein have the same meaning as commonly understood by one skilled in the art to which this subject matter belongs.

Where a range of values is provided, it is understood that each intervening value, to the tenth of the unit of the lower limit, unless the context clearly dictates otherwise, between the upper and lower limit of that range, and any other stated or intervening value in that stated range, is encompassed within the subject matter described herein. The upper and lower limits of these smaller ranges may independently be included in the smaller ranges, and are also encompassed within the subject matter described herein, subject to any specifically excluded limit in the stated range. Where the stated range includes one or both of the limits, ranges excluding either or both of those included limits are also included in the subject matter described herein.

It must be noted that as used herein and in the appended claims, the singular forms “a,” “and,” and “the” include plural referents unless the context clearly dictates otherwise.

FIG. 1 depicts a handheld pupillometer, such as that described in U.S. Pat. No. 7,147,327. Pupillometer 10 includes a display 12, handle 15 for gripping the pupillometer while using it, and headrest 16. Headrest 16 is used to position the pupillometer 10 optimally to take measurements of the pupils of an individual's eyes. U.S. Pat. No. 7,216,985, which is incorporated herein by reference in its entirety, describes headrests of the type that may be used with pupillometer 10. Pupillometer 10 also includes a keypad 14, including various control buttons including buttons 14a and 14b, which are used to operate the pupillometer 10.

Pupillometer 10 operates essentially as a handheld optical scanner. In one embodiment, it stimulates the eye of an individual with a flash of light and captures and analyzes a rapid sequence of digital images to obtain a temporal measurement of the diameter of the individual's pupil. The intensity and duration of the light stimulus can be set by the user of pupillometer 10 using the controls in the keypad 14. Pupillometer 10 can acquire images using a self-contained infrared illumination source and a digital camera. It analyzes the captured image data and displays a summary of the measurement in the display 12, which can be an LCD display. Data may also be printed out on an optional thermal printer or downloaded to an external computer via an infrared port (IrDA) or transmitted via USB port and cable or wirelessly through Bluetooth, wifi, RF or other known means to a computer. Pupillometer 10 can use a menu driven graphical user interface with a color LCD screen 12 for data display. A keypad 14 completes the user interface and enables manual entry of individual subject identification (ID) numbers and other information.

Pupillometer 10 can be powered by any number of power sources known to those of skill in the art. In one embodiment it is powered by a 4.2 volt rechargeable lithium ion battery.

Pupillary data sampled at approximately ten frames per second (10 fps) (or less), approximately twenty frames per second (20 fps), approximately thirty frames per second (30 fps), approximately forty frames per second (40 fps), approximately fifty frames per second (50 fps), or more and for a total duration of up to about 3600 seconds (i.e., about 60 minutes) can be used in the calculation of a number of different pupillary reaction variables that can be displayed numerically or graphically on the display 12 at the end of each measurement. In one embodiment, the Pupillometer 10 takes one measurement of a pupil by sampling thirty frames per second (30 fps) for three seconds to acquire a total of approximately ninety sequential images of a pupil's response to a stimulus. Pupillometer 10 can sample data at anywhere between about 1 frame per second (1 fps) (or less) to about one hundred frames per second (100 fps) (or more) for any length of time between about one (1) second (or less) and about 3600 seconds (i.e., about 60 minutes).

Pupillometer 10 has internal memory large enough to store three thousand or more measurements. All measurement data contained in Pupillometer's 10 memory can be downloaded to an external computer or laptop, such as a Window® based computer or laptop or computer or laptop with a different operating system. Measurement data can be encrypted and contained in a file named with the date and time of the moment of the download and extension “dat”. For example, R_20080909_1030.dat would indicate a file downloaded on Sep. 9, 2008 at 10:30. Previous measurements and data can be browsed, retrieved and printed using the keypad controls 14 and appropriate menus displayed in the screen display 12.

Pupillary measurements can be divided into three phases as shown in FIGS. 3A-3C. Pressing either one of the buttons 14a (for the right pupil) or 14b (for the left pupil) on the keypad 14 can activate the first phase, i.e., the targeting phase of pupillometer 10. By pressing key 14a, pupillometer 10 automatically saves the data as left eye data, and by pressing key 14b, pupillometer 10 automatically saves the data as right eye data. In one embodiment, the operator of pupillometer 10 holds down button 14a or 14b during the targeting phase while keeping pupillometer 10 snuggly up to the subject's eye. During the targeting phase, a video image of the eye is displayed in the LCD window as shown in FIG. 3A. The subject's pupil must be centered within the field of view as shown in FIG. 3A.

During phase 2 of pupillary measurement, pupillometer 10 has a pupil finder that automatically detects the pupil 20 and marks it with a perimeter 25 drawn around the perimeter of the pupil 20. Algorithms and software for detecting a pupil and drawing a perimeter around it are known in the art. For example, U.S. Pat. No. 7,147,327, describes various imaging processing procedures and methods that can be used to do that. The perimeter 25 can be any color that is easy to visualize, such as green, white, red, etc. From the beginning of phase 1 until now, the operator has been holding down the button 14a or 14b.

Once the pupil finder of pupillometer 10 has found the pupil 20 and has marked it with the perimeter 25, the operator can now release the button 14a or 14b. Release of the button 14a or 14b initiates the third phase, which is the actual measurement phase. In the measurement phase, pupillometer 10 subject's the individual's eye to a flash of light applied at time 0.0 seconds. In other words the flash of light is applied at the same instant as the first image of the pupil is being recorded. The flash of light can also be applied just before or just after the pupillometer begins recording images of the pupil. As discussed above, the intensity and duration of the flash of light can be controlled by the operator. FIG. 3C shows an image on the display 12 of pupillometer 10 of the pupil as it is being recorded during the measurement phase. The pupillometer may also be set to operate without the flash of light, such that measurements are taken of a pupil that has not been stimulated but is at rest. When the comparison of the left and right pupil is made for measurements that don't involve a stimulus, the videos of the right and left pupil are synchronized in the same manner as when measurements are taken after a stimulus.

FIG. 2 shows a screen shot of the display 12 of pupillometer 10 showing a results page that includes various measurements of the pupil that was subjected to the light stimulus and recorded during phases 1-3 described above. The results page can show a plot of the pupil response and can, for example, report the following information:
ID, the identification code of the subject measured (for example 0 in FIG. 2); which eye was measured (right in the example in FIG. 2), and the date and time of the measurement. If the measurement was affected by certain problems during phases 1-3, the data shown in the results page may be in a special color, such as red, to indicate that there was a problem. Some examples of problems that can occur during measurement are excessive eye blinks or excessive pupillometer motion.
The intensity and duration of the light stimulation. In the example shown in FIG. 2, the light stimulation intensity was 180 micro Watts for a total duration of 667 milliseconds. The duration of the light stimulation is also represented by a colored vertical line superimposed over the pupil plot (0.0 seconds is always the onset of the light stimulation).
MAX and MIN represent the diameter of the pupil before the constriction (MAX=2.8 mm in this example) and just at the peak of the constriction (MIN=2.2 mm in this example), respectively in FIG. 2. These measurements are given in millimeters and are represented by two horizontal gray lines superimposed over the pupil plot. The variable CON is the percent of the constriction (MAX−MIN)/MAX as a percent.
LAT is the latency and it represents the amount of time it takes before the onset of pupillary constriction in response to the light stimulus. It is given in seconds (for example 0.23 seconds in the FIG. 3) and is represented by a vertical colored line, different from the color of the light stimulation duration line, superimposed over the pupil plot.
ACV and MCV are the average and the maximum constriction velocity and they are given in millimeters/second, respectively. The negative sign differentiates the constriction from the opposite papillary movement that is dilation. Both velocities refer to the constricting movement of the pupil diameter responding to the flash of light.
ADV is the dilation velocity (given in millimeters/second) and represents the average pupillary velocity, when, after having reached the peak of the constriction, the pupil tends to recover and to dilate back to its initial resting size. Pupillary recovery after a light reflex constriction is usually characterized by an initial and more rapid phase followed by a much slower converging movement. The dilation velocity reported here refers to the initial and stronger recovery and is indicated in the graph in FIG. 3 by a line fit to the corresponding dilation phase of the pupil profile.
T75 is the total time taken by the pupil to constrict and then to return to 75% of the initial resting pupil size. It is given in seconds and is represented by a vertical colored line superimposed on the pupil plot in a color that is different than the other lines that are superimposed on the pupil plot.

The above describes operation of pupillometer 10 with respect to each measurement of a pupil that is taken. When taking a measurement of a pupil, the operator uses the keypad 14 to enter various data regarding the pupil. That data can include, e.g., the identity of the individual whose pupil it is. As discussed above, pupillometer 10 memory stores that information and can use it to perform direct comparisons between the response of the left eye and the response of the right eye in paired measurements to the flash of light applied by pupillometer 10. Which pupil of the individual is being measured is specified by the keypad buttons 14a and 14b. If the user presses keypad button 14a then the pupillometer automatically stores the pupillary measurement as left eye data, and if the user presses keypad button 14b then the pupillometer automatically stores the pupillary measurement data as right eye data.

In one embodiment, pupillometer 10 has the following components that enable it to perform a comparison between the left and right eyes of the individual. Pupillometer 10 has a display 12 that is sized to simultaneously display a video of y or more seconds in length of a left pupil and a video of y or more seconds in length of a right pupil of the same individual. The lengths of each video can be anywhere from 1 second or less to sixty or more minutes. Pupillometer 10 has an imaging apparatus that includes a pupil finder that identifies the perimeter of a pupil and a microprocessor. The imaging apparatus is capable of recording images of an individual's pupils at a rate of x frames per second for a period of y or more seconds and playing back said images as a video at x frames per second or at another rate that is faster or slower than x frames per second. As explained above, in one embodiment, the imaging apparatus records or samples the images at a rate of approximately 30 fps for a period of approximately 3.0 seconds. The playback mechanism of pupillometer 10 can play the videos back on display 12 at 30 fps, or at a different rate controlled by the user using the keypad 14 to control the playback rate. The playback mechanism can also pause the video playback or it can fast-forward or rewind the video playback at various speeds controlled by the user using the keypad 14 controls, which can include pause, rewind, fast-forward and video playback, fast-forward and rewind speed buttons and/or functions. For example, the user can view the images one image set at a time and can control how long he or she wants to view the image set before manually forwarding or advancing to the next set of images or rewinding to the previous set of images. This can be done to compare the right pupil to the left pupil image set by image by set for each set of images taken during the procedure. The user can pause as long as he or she wants at a particular set of images to, for example, compare the difference between the right pupil and the left pupil in that set of images.

Pupillometer 10 also has a memory in communication with the microprocessor. The memory has stored therein a pupil comparison program. The pupil comparison program can include a pupil finder as described above. The pupil comparison program enables the microprocessor to perform the following functions:
(i) identify a center of the left pupil and a center of the right pupil for each image frame;
(ii) synchronize each frame of the two videos starting from the first frame;
(iii) cause the display to display the two videos simultaneously such that each of the frames of the video of the left eye is synchronized to a corresponding frame of the video of the right eye when played back on the display;
(iv) cause the two videos to be displayed so that the center of the left pupil in each frame is aligned on the display with the center of the right pupil for the corresponding frame.

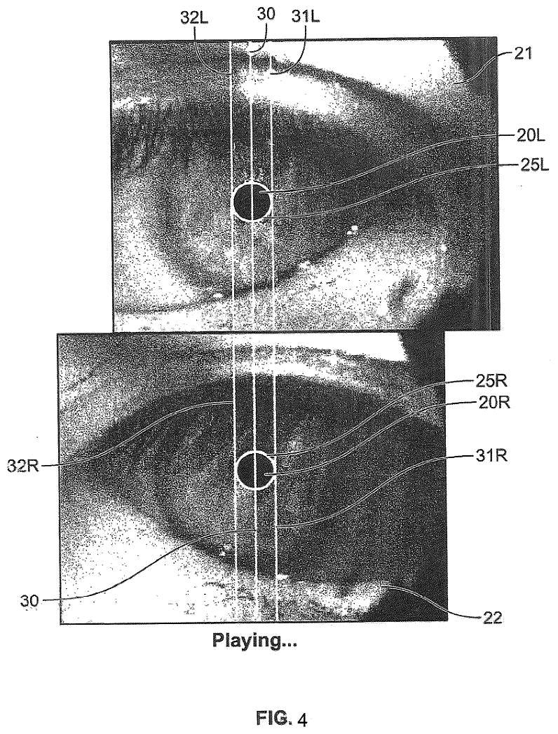

FIG. 4 shows one example of a pupillometer display 12 showing an image frame 21 of a left pupil 20L recorded during measurement of the pupil as described above, and an image frame 22 of a right pupil 20R recorded during measurement of the pupil as described above. Image frame 21 is part of a series of sequential image frames of the left pupil recorded at a rate of x frames per second and which together form a video of y seconds in length. In one embodiment, x is approximately 30 and y is approximately 3.0. In other embodiments, x can be about 10 (or less), about 20, about 40, about 50 or more and y can be 1, 2, 4, 5 or up to 3600 or more, and in general x can be between about 1 (or less) and about 100 frames (or more) per second, and y can be any length of time between about one (1) second and 3600 seconds (i.e., 60 minutes) (or more). Image frame 22 is part of a series of sequential image frames of the right pupil recorded at the same rate of x frames per second and which together form a video of y seconds in length, which is the same length as the video associated with image frame 21.

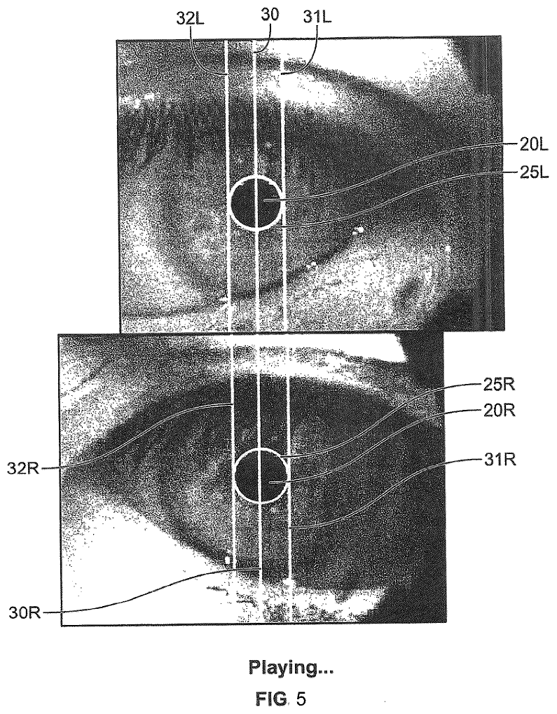

Thus, in one embodiment, there are approximately 90 image frames for the video of the left pupil and 90 image frames for the video of the right pupil. Each image frame of the video of the left pupil has a corresponding image frame in time in the video of the right pupil. For example, image frame 1 of the left pupil and image frame 1 of the right pupil are both taken at the exact same amount of time after onset of the flash of light from pupillometer 10 (alternatively, image frame 1 can be taken just before or just after onset of the flash of light). Thus, image frame 1 of the left pupil and image frame 1 of the right pupil are corresponding image frames. Likewise, image frame 2 of the left pupil and image frame 2 of the right pupil are both taken at the exact same amount of time after onset of the flash of light from pupillometer 10 (or from onset of video recording without a flash of light). Alternatively, image frame 2 of each pupil can be taken just before or just after onset of the flash of light. Thus, image frame 2 of the left pupil and image frame 2 of the right pupil are corresponding image frames, and so on and so forth for approximately 90 images that when played back at 30 fps form a video that lasts about 3 seconds. In the example shown in FIG. 5, image frame 21 is the 24th image of the left eye taken by pupillometer 10, which means that it is taken at 800 milliseconds after onset of the flash of light from pupillometer 10 (or from onset of video recording without a flash of light). Likewise, image frame 22 is the 24^{th} image of the right eye taken by pupillometer 10, which means that it too is taken at 800 milliseconds after onset of the flash of light from pupillometer 10 (or from onset of video recording without a flash of light).

It should be understood that with respect to all embodiments described herein, the first image of each pupil can be taken just before, simultaneously with, or just after the onset of the flash of light. Thus, for example, the first image of each pupil can be taken within one, two, three, four, five, six, seven, eight, nine or more milliseconds up to one second before or after the onset of the flash of light. It can also be taken simultaneously with the flash of light. In one embodiment, the first image of each pupil is taken 500 milliseconds before the onset of the flash of light.

As shown in FIG. 4, the pupil comparison program enables the microprocessor to determine the center of each pupil and to draw a straight line 30 that runs through the center of each pupil. That way pupils 20L (left pupil) and 20R (right pupil) are lined up with one another on the display. Pupil centering programs are known in the art and some are described in U.S. Pat. No. 7,147,327. FIG. 4 shows just one frame of each pupillary measurement of the left pupil 20L being lined up with its corresponding pupillary measurement frame of the right pupil 20R, but each frame of the pupillary measurement of the left pupil 20L is lined up with its corresponding pupillary measurement frame of the right pupil on the display 12. Thus, when the two videos are played back on the display 12 simultaneously, it is easy to see the difference in response of the left pupil relative to the right pupil. One of the reasons why it is so important to align and center the two pupils in the video playback mode is to compensate for eye movement so that the operator can easily compare one eye to the other.

The pupil comparison program provides another feature that makes it even easier to follow the difference in the pupillary responses of the left and right pupil. The pupil comparison program enables the microprocessor to draw a pair of parallel straight lines that extend from the perimeter of the left pupil to the perimeter of the right pupil in each image frame. As shown in FIG. 4, the parallel lines have a first section 32L/31L and a second section 32R/31R, wherein in the first section 32L/31L the distance between the parallel lines is defined by the diameter of the left pupil 20L for each frame, and in the second section the distance between the parallel lines 32R/31R is defined by the diameter of the right pupil 20R for each frame. The diameter of the left pupil 20L is defined by the pupil perimeter 25L, which is established by pupillometer 10 using its pupil finder. The diameter of the right pupil 20R is defined by the pupil perimeter 25R, which is likewise established by pupillometer 10 using its pupil finder. In order to further make it easier to visualize the difference in response between the left and right eyes, the first section lines 32L/31L can be a different color than the second section lines 32R/31R. For examples parallel lines 32L/31L can be red while parallel lines 32R/31R can be green.

FIG. 5 shows the 72^{nd} image frame of each video of each eye, which means that each frame was taken at 2.4 seconds after onset of the flash of light of pupillometer 10. At 2.4 seconds after onset of the flash of light, the pupils 20L and 20R have dilated back to their resting position. Looking at the still images in FIGS. 4 and 5, it would appear that there is very little pupil size asymmetricity in this individual. But for a better understanding, one would view the video comparing the two sets of images at 30 fps or perhaps at a slower speed such as 10 fps to get a much clearer view of whether or not there is substantial anisocoria or asymmetry in pupillary response to light stimulus. The pairing of parallel lines 32L/31L with 32R/31R makes it very clear whether there is anisocoria or asymmetry in papillary response to light stimulus.

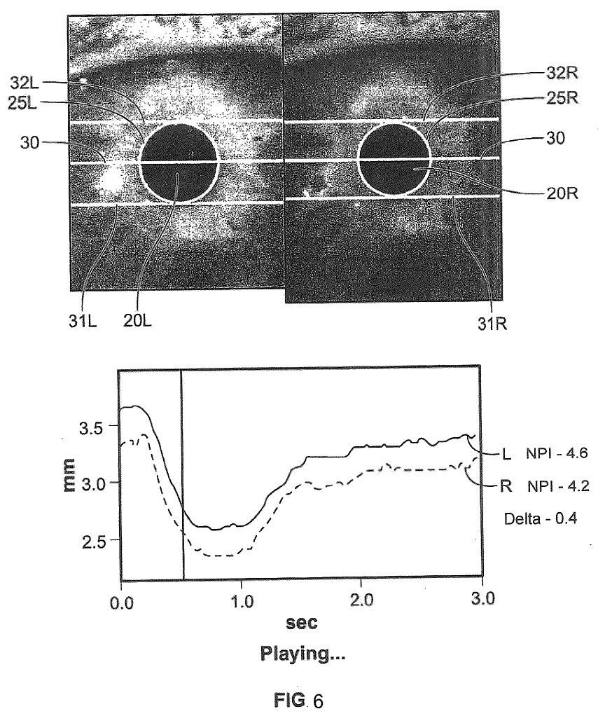

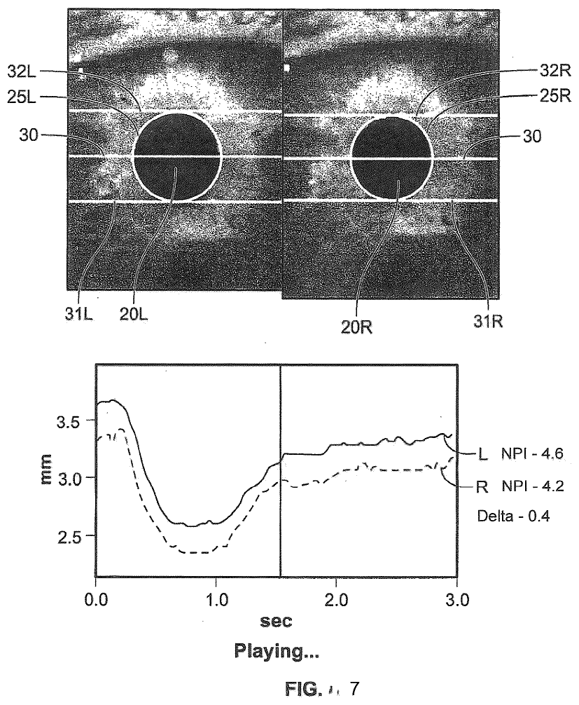

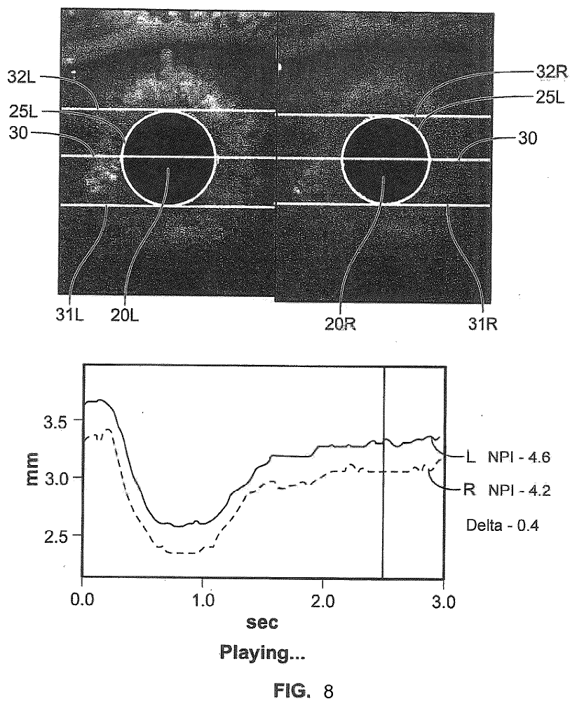

FIGS. 6-8 show a further feature of pupillometer 10. This further feature allows the user to see on the display 12 of pupillometer 10 not just synchronized and centered videos of the left and right pupil at the same time, as described above, but to view a graphical representation of the pupillary response of the left and right pupils superimposed together on a single graph. Images in FIG. 6 are taken at about 600 milliseconds after onset of the flash of light from pupillometer 10 (or from onset of video recording without a flash of light) as the pupils 20L and 20R are dilating. There is some minor difference as depicted in the graph below the images of the pupils 20L and 20R and as shown by the disconnection between sections 32L/31L and 32R/31R of the parallel lines. Images in FIG. 7 are taken at about 1.5 seconds after onset of the flash of light from pupillometer 10 (or from onset of video recording without a flash of light) as the pupils 20L and 20R are recovering back to their resting state. There is again some minor difference as depicted in the graph below the images of the pupils 20L and 20R and as shown by the disconnection between sections 32L/31L and 32R/31R of the parallel lines. Images in FIG. 8 are taken at about 2.6 seconds after onset of the flash of light from pupillometer 10 (or from onset of video recording without a flash of light) as the pupils 20L and 20R have fully dilated back to their resting position. Again, there is some minor difference as depicted in the graph below the images of the pupils 20L and 20R and as shown by the disconnection between sections 32L/31L and 32R/31R of the parallel lines. If viewed in rapid succession at 30 fps or even if slowed down to 10 fps, the operator of pupillometer 10 would be able to clearly see the difference in pupillary response between the left and right pupils to the flash of light from pupillometer 10 (or from onset of video recording without a flash of light). For example, the user can view the images one image set at a time and can control how long he or she wants to view the image set before manually forwarding or advancing to the next set of images or rewinding to the previous set of images. This can be done to compare the right pupil to the left pupil image set by image by set for each set of images taken during the procedure. The user can pause as long as he or she wants at a particular set of images to, for example, compare the difference between the right pupil and the left pupil in that set of images.

A pupillary index of each eye of a paired measurement can also be displayed along with the graphical data for that eye as shown in FIGS. 6-8. A pupillary index can be a value (displayed in different formats including alphanumerical value, color, graph, waveform, symbol, sound (the pupilometer would include a speaker or other means of generating sound), etc.) that is derived from processing different characteristics of a pupil's response to a stimulus, such as a light stimulus, and calculating a pupillary index value. The different characteristics of a pupil's response to a stimulus can include minimum size (e.g., diameter or circumference), maximum size (e.g., diameter or circumference), percentage change in size, average constriction velocity, average dilation velocity, maximum constriction velocity, maximum dilation velocity, latency of pupillary light response, and other dynamic response characteristics. One type of pupillary index is “NPI” developed by Neuroptics®.

As shown in FIGS. 6-8, pupilometer 10 can calculate NPI of the right eye after it has been measured and display it along with the images and data for the right eye, and then also calculate NPI of the left eye after it has been measured as part of a paired measurement and display it along with the images and data for the left eye. Thus, as shown in FIGS. 6-8, the user can get a comprehensive understanding of the patient's pupillary response and the patient's potential health or neurological outcome by seeing on one screen the paired pupillary response of both eyes in graphical form, alphanumeric index form (NPI) and video image form all at once. The two NPI's can be compared manually by the healthcare provider in real time while being able to visualize any anisocoria between the two eyes both in visual and graphical form. This feature provides the user with a comprehensive understanding of the patient's pupillary response and the potential outcome of the patient.

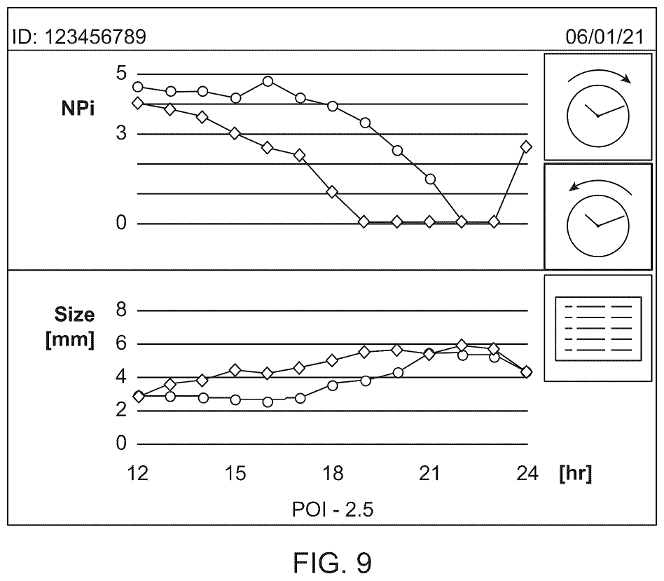

In another embodiment, the two NPI's can be compared by the pupilometer and an overall final patient outcome index (“POI”) can be calculated and displayed. The POI can take into account the difference between NPI of the left eye and NPI of the right eye as one factor or element to determine POI. POI is an index that predicts the health or neurological outcome of the patient. FIG. 9 shows a screen shot depicting NPI differential between left and right eyes in graphical form over time along with POI. POI changes with each measurement. For example, POI may indicate a bad outcome after a first paired measurement of the left and right pupils. However, after the second paired measurement, POI may indicate a neutral outcome, and after the third measurement, POI may indicate a favorable outcome. POI may be provided in the same manner as NPI, as an alphanumeric value, a color, a symbol, a graph or waveform or other visual or sound representation. A single POI can be displayed on the display after each paired measurement of the pupils. Alternatively, a POI graph can be displayed along with the NPI and size graphs shown in FIG. 9, in which POI values are graphically tracked along with the NPI and size values.

Methods for monitoring asymmetry in response between the left and right eye are also described herein. The methods are directed to whether or not an individual has anisocoria and the level and severity of anisocoria, or asymmetry in response between the left and right eye to a stimulus, such as a light stimulus or other stimulus (or no stimulus at all). Determining whether a patient has anisocoria or asymmetry in response can have diagnostic value.

In one aspect, a method for monitoring asymmetry in response to a stimulus between a left pupil and a right pupil of an individual involves the following steps, which can be performed with the aid of pupillometer 10 described above and illustrated in FIG. 1, or other diagnostic equipment. An operator, usually a medical professional, will position pupillometer 10 with the headrest 16 against the patient's face around her left eye with the body of pupillometer 10 being perpendicular relative to the patient's axis of vision as shown in FIG. 2. It does not matter which eye is analyzed first as long as the data regarding whether the pupillary measurement is of the left eye or right eye is entered prior to taking the pupillary measurement. Pupillometer 10 is activated as described and it is used to record for a period of time y at a rate of x image frames per second a response of the left pupil to a flash of light (or simply recording the pupil at rest without a flash of light). When the measurement phase is activated, pupillometer 10 will issue a flash of light in the field of view of the left eye (or simply recording the pupil at rest without a flash of light) and will simultaneously begin recording images of the left pupil for a period of time y at a rate of x image frames per second as noted above. Recording will automatically stop after the period of time y has elapsed, and the images will be stored in the memory of pupillometer 10. The result of the recording is a video made up of a number of sequential image frames that when played back depict in a movie format the response of the pupil to the flash of light (or simply recording the pupil at rest without a flash of light). The same steps are then performed on the right eye and the image data of the right eye is also stored in the memory of pupillometer 10.

Now the operator can use the pupil comparison feature of pupillometer 10 to display on the display screen 12 of pupillometer 10 simultaneously the first set of image frames of the response of the left pupil and the second set of image frames of the response of the right pupil to the flash of light (or no stimulus at all). This will initiate the playback feature on the pupillometer 10. The pupil comparison feature of the pupillometer 10 will arrange the two sets of image frames on the display so that the center of the left pupil is aligned with the center of the right pupil on the display and the two sets of image frames are synchronized per frame starting from the first frame for each set with a straight line running from the center of both pupils. Stated somewhat differently, the two videos, the video of the left pupil and the video of the right pupil, will be aligned on the display 12 so that the center of the left pupil is aligned with the center of the right pupil on the display 12 and pupillometer 10 will then play the two videos simultaneously and in a synchronized fashion on the display screen 12.

To further enhance the ability of the operator to perceive any anisocoria, the comparison feature of pupillometer 10 draws a pair of parallel straight lines that extend from the perimeter of the left pupil to the perimeter of the right pupil in each image frame. The parallel lines have a first section and a second section, wherein in the first section the distance between the parallel lines is defined by the diameter of the left pupil for each image frame, and in the second section the distance between the parallel lines is defined by the diameter of the right pupil for each image frame. The color of the parallel lines in the first section can be different than the color of the parallel lines in the second section to make it easier for the operator to perceive any anisocoria. For example, the parallel lines in the first section can be green and the parallel lines in the second section can be red or vice versa. In addition, the user can view the images one image set at a time and can control how long he or she wants to view the image set before manually forwarding or advancing to the next set of images or rewinding to the previous set of images. This can be done to compare the right pupil to the left pupil image set by image by set for each set of images taken during the procedure. The user can pause as long as he or she wants at a particular set of images to, for example, compare the difference between the right pupil and the left pupil in that set of images.

In another embodiment, pupillometer 10 can be binocular (not shown) so that measurements of the left and right eye are taken simultaneously. Binocular Pupillometers, such as Neuroptics'® DP-1000 and DP-2000 Pupillometers for Research are well known in the art.

In accordance with another embodiment, applicants have made the unexpected discovery that a differential in a pupillary index generated from taking paired measurements of a left and right eye can be indicative of a neurological abnormality and can be used to predict the health or neurological outcome of the patient, even if both pupillary indexes of the paired measurement are within the normal range. An example of a “paired measurement” is the following: using a pupillometer to take a measurement of a left eye to a light stimulus; promptly following measurement of the left eye, using the same pupillometer to take a measurement of the right eye to the same light stimulus (meaning the light stimulus provided to the left eye has the same characteristics or attributes as the light stimulus provided to the right eye, e.g., same brightness, color, amplitude, magnitude, and duration of light). The left and right eye can be measured in any order so long as the measurements are taken close in time during the same examination and the light stimulus is the same for each eye.

For example, using the Neuroptics® NPI®-200, applicants have discovered that a differential in NPI generated from a paired measurement, even if both NPIs are between 3.0 and 4.9, between the left and right NPI of an individual is indicative of a neurological abnormality and can be predictive of the health or neurological outcome of the patient. This was verified by looking at the modified Rankin Score at discharge (DC mRS) of patients admitted to neuroscience intensive care units with different types of acute brain injuries. Acute brain injury as used herein includes patients having suffered a stroke (including subarachnoid hemorrhage, intracerebral hemorrhage, arterial ischemic stroke, and aneurysm), and traumatic brain injury (including subdural hematoma). It was found that patients with at least one occurrence of NPI differential during their stay in the ICU have poorer outcomes (higher DC mRS, 4.1 vs. 2.7, P<0.001) than those with no differentials, even when both pupils were normal with all NPI's>3.0. Thus, NPI differential, not only the monocular assessment of the NPI, is an important factor that clinicians should consider when managing hospitalized brain injury patients.

In one embodiment, the pupilometer can also store multiple results for each patient and each eye of each patient in the memory of the pupilometer and display them on its display (such as display 12) in a list format, a summary format, or as a graph (as shown in FIG. 9). In list format, the pupilometer can display one or multiple results of pupillary index and differential of pupillary index on the display. For example, the display can show a list of paired scans or measurements showing the NPIs of the left eye, the NPIs of the right eye, and the difference in the NPI's (“NPI differential”) for each paired scan, thus providing the healthcare professional with a quick view of the NPI differentials for a number of scans. When the differential is greater than a threshold differential, the differential can be highlighted for ease of reference and also to bring attention to the excessive NPI differential (e.g., scan 2 of TABLE A below). An example of a list format displaying five scans (although the display can display two, three, four, six, seven, eight, nine, ten or more scans) by the display of the pupilometer is as follows:

In one embodiment, an NPI differential of 0.7 or more is a threshold differential that is predictive of an unfavorable health or neurological outcome for a hospitalized acute brain injury patient. Thus, any NPI differential that equals or exceeds 0.7 is highlighted so as to bring attention to the excessive NPI differential, providing the healthcare professional with a tool to predict the health or neurological outcome of a hospitalized acute brain injury patient. The table can also include a column showing the time at which the scan was performed. Such a table depicted on the display of a pupilometer provides the user with an easy way to identify a differential that is greater than a threshold differential even when the NPIs are in the normal range.

The pupilometer display can also display the results of the pupillary measurements or scans in summary format as well. An example of a summary format showing the results of three hundred sixty paired scans or measurements of the left and right eyes (although this can be as few as two or as many as several thousand) displayed by the display of the pupilometer is as follows:

TABLE B

| Summary | Right Eye | Left Eye |
|---|---|---|
| NPI ≥ 3 | 260 | 310 |
| NPI < 3 | 50 | 30 |
| NPI = 0 | 50 | 20 |
| ΔNPI > 0.7 | 40 | 10 |

This above exemplary summary format indicates that three hundred sixty paired scans or measurements were taken of the left and right eyes of a subject, such as a hospitalized acute brain injury patient. Each time the left eye was measured, the right eye was also measured either immediately before or after the left eye, and this procedure was followed three hundred sixty times. Of the three hundred sixty measurements taken of the right eye, two hundred sixty of those measurements resulted in NPI of ≥3 (i.e., normative range), fifty of those measurements resulted in NPI of <3 (i.e., abnormal response), and fifty of those measurements resulted in NPI showing no pupillary response at all. Of the three hundred sixty measurements taken of the left eye, three hundred ten of those measurements resulted in NPI of ≥3 (i.e., normative range), thirty of those measurements resulted in NPI of <3 (i.e., abnormal response), and twenty of those measurements resulted in NPI showing no pupillary response at all. In addition, of the three hundred sixty measurements taken of the left and right eyes successively, there was a greater than or equal to threshold difference in NPI between the left and right eyes fifty times, and of those fifty times, the right eye had a lower NPI than the left eye forty times, and the left eye had a lower NPI than the right eye ten times. This means that three hundred ten times, the difference in NPI between the left eye and right eye was less than a threshold differential and fifty times it was greater than or equal to a threshold value. In some embodiments, NPI differential of 0.7 is used as the threshold differential between the left and right eyes for predicting an unfavorable outcome in a brain injury patient, including, a hospitalized acute brain injury patient.

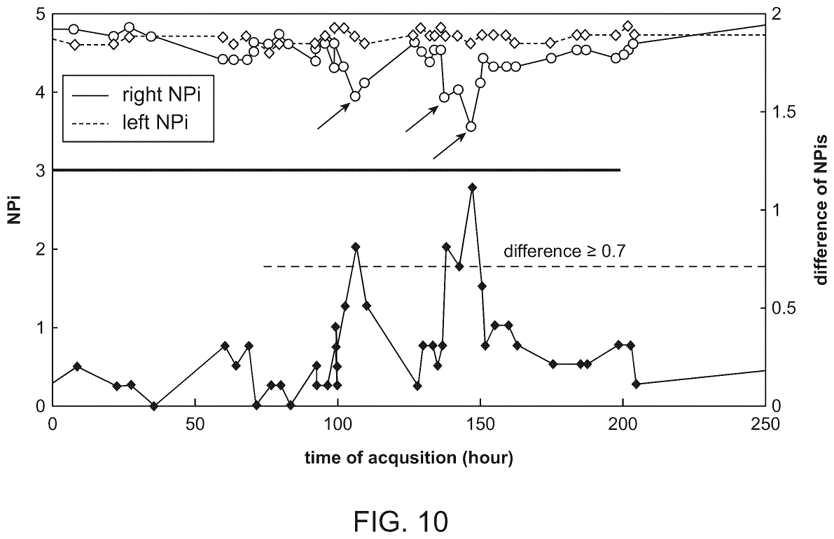

The pupilometer display can also display the results of the pupillary measurements or scans in graphical format as well. One example is a graph that shows the paired NPIs of the left and right eye over time with the precise differential also displayed on the same graph along with the threshold differential depicted on the same graph. FIG. 10 provides a graph that can be displayed on the display of a pupillometer, as shown on the display of pupillometer 10 of FIG. 1. It is a demonstration of a graphical representation of the paired NPIs of left and right eyes of a subject relative to one another over time with NPI differential and threshold cutoff values also plotted on the graph. The x axis shows the time of acquisition of the paired measurements of the pupils in response to a light stimulus, the left y-axis shows NPI (value of 0-4.9) for each paired measurement, and the right y-axis shows the differential in NPI at each paired measurement, with a line drawn across the graph at a threshold differential of 0.7. Displaying this graph on the display 12 of pupillometer 10 provides a useful tool for practitioners to predict the outcome of their patients based on paired NPI values and their differentials.

In one embodiment, a program product is provided. The program product includes a computer-readable medium and computer-executable instructions recorded on the computer-readable medium for performing a method of predicting the outcome of a patient, the method including: analyzing a series of images or a video recording of a left pupil of a hospitalized acute brain injury patient responding to a stimulus such as a flash of light; generating a pupillary index that indicates whether the response of the pupil is normal, abnormal or non-responsive; causing a digital display such as a computer monitor, smart phone or display of a pupilometer to display the pupillary index of the left pupil; analyzing a series of images or a video recording of a right pupil of said patient to another stimulus having the same attributes or characteristics as the first stimulus; generating a pupillary index that indicates whether the response of the right pupil is normal, abnormal or non-responsive; causing the digital display to display the pupillary index of the right pupil; comparing the pupillary index of the left pupil with the pupillary index of the right pupil and calculating the differential between the two indexes; and causing the digital display to display the differential. The program product can also cause the display to display a graph such as the one shown in FIG. 10. A pupillary index differential that equals or exceeds the threshold pupillary index differential 0.7, as shown on the graph in FIG. 10, is a predicter of an unfavorable health or neurological outcome for the hospitalized acute brain injury patient.

In another embodiment, a pupilometer can include an algorithm that calculates the differential in NPI between the left and right eye and generates a final overall POI after each differential measurement. This final overall POI can be predictive of the health or neurological outcome of the patient. One component of the pupilometer can be an algorithm or software application that causes a digital display, such as the digital display of the pupilometer, a computer monitor, or smart phone display, to graphically display the paired neurological indexes generated for a left eye and right eye, and the differential in neurological indexes between the left and right eyes including each instance when the neurological indexes of the left and right eye meet or exceed a given threshold differential in neurological index. The information can be displayed in graphical form, such as FIG. 10.

In another embodiment, the algorithm can further cause a digital display to display a table, such as TABLE B herein, that includes the number of times a left pupil had an abnormal pupillary response to a stimulus such as a light stimulus, the number of times a right pupil had an abnormal pupillary response to the same stimulus, the number of times a right pupil had a normal pupillary response to the same stimulus, the number of times a left pupil had a normal pupillary response to the same stimulus, and the number of times the difference between the pupillary response of the light eye and the pupillary response of the right eye was greater than a threshold differential. The pupillary response data can be depicted as a pupillary index such as NPI, as shown in TABLE B.

In accordance with another embodiment, applicants have made the unexpected discovery with data collected over several years of research that persistence of differential in NPI between the left and right eye in a hospitalized subject is an indicator of neural worsening or worse patient outcomes in patients with stroke, TBI, acute brain injury or other neurological impairments even when the difference in NPI is less than 0.7 between the left and right eye. In other words, when there is a persistent difference in NPI between the left and right eye, even when that difference is less than 0.7, it is an indicator of worse outcomes in patients that are hospitalized. This condition is referred to as NPI differential persistence. NPI differential persistence is defined as three or more sequential measurements of NPI over any period of time in which there is a difference in NPI between the left and right eyes even if that difference is less than 0.7. NPI differential persistence is not limited to a specific number of consecutive measurements; it can be three or more and may occur at different time scales (e.g., every hour, every two hours, etc.). A finding that NPI differential persistence is an indicator of neural worsening or greater disability at hospital discharge in patients who have suffered a stroke, TBI, acute brain injury or other neurological impairment, was not expected, because prior to the revelation of the data, it was believed that only NPI scores below 3.0 were an indicator of neural worsening. However, it was discovered that not only NPI differential of greater than 0.7 as explained above is an indicator of neural worsening in such patients, but the data here unexpectedly demonstrates that NPI differential persistence is also an indicator of neural worsening or greater disability at hospital discharge in patients who have sufferance a stroke, TBI, acute brain injury or other neurological impairment.

The bar plot set forth below summarizes the data collected from approximately nine thousand hospitalized subjects over a number of years and includes patients with various brain impairments conditions, such as stroke, TBI, acute brain injury and other neurological impairments. The bar plot shows the mean DC mRS, which stands for modified Rankin Scale, a widely used clinical tool for measuring the degree of disability at hospital discharge in patients who have suffered a stroke, TBI, acute brain injury or other neurological impairments. The higher the DC_mRS, the poorer the outcome. As shown in the bar plot, patients with Differential (2), Persistency (3), i.e., NPI differential persistence, or NPi=0 (4) all have worse outcomes than patients with no NPI abnormalities.

In one embodiment, a method of assessing the neurological condition of a hospitalized patient includes determining whether the patient exhibits NPI differential persistence. A patient that does exhibit NPI differential persistence may be monitored more closely or be recommended for a longer hospital stay than would otherwise be recommended.

All of algorithms or software applications described herein can be independent components of a pupilometer or they can be stand-alone applications that can be executed by a computer, a smart phone, or a pupilometer.

### Example 1

The Neuroptics NPI-200 is a monocular, handheld, and battery-operated pupillometer based on infrared technology, a proprietary optics apparatus and pupil tracking image processing algorithms that automates the entire process of pupil assessment in a simple, rapid, “point-and-scan” procedure. One component of the Neuroptics NPI-200 is an algorithm that generates a Neurological Pupillary Index (“NPI”) as previously described. NPI is correlated to outcome and diagnosis in patients with traumatic brain injury, stroke, or cardiac arrest¹⁻⁵, and is not influenced by sedation or mild hypothermia^{4,6}. Despite the significant volume of literature about NPI, its differential seems to have been overlooked by investigators and clinicians in general until now.

### Methods

END-PANIC is an international ongoing multicenter prospective registry of pupillary measurements (NCT02804438). Following Institutional Review Board approval, data from this registry used in our analysis were collected between March 2015 and January 2021 from patients with a variety of neurological conditions admitted to neurocritical care units at 1 Japanese and 4 U.S. hospitals^{6,9}. Outcome is represented by the modified Rankin Score at discharge (DC mRS) and analyzed as a function of NPI and NPI differential. The DC mRS ranges between 0 and 6, with higher values corresponding to more severe disability and worse outcomes; a score of 6 is assigned to death.

For each patient, pupillometry was usually performed several times a day for the entire intensive care unit (ICU) length of stay using the NPI-200 pupillometer (NeurOptics Inc.) which provides AIP using a monocular, handheld, and battery-operated infrared technology^{7,10}. Each observation is comprised of two consecutive measurements: one for the right pupil and one for the left pupil. Observations are automatically time and date stamped. For the analysis, we considered each patient's minimum (most abnormal) NPI score recorded during their ICU stay and, for NPI differential, the largest difference between all their pairs of NPI's.

The END-PANIC registry includes patient demographics, primary diagnosis, and results on several neurological scales including DC mRS. Only patients with a primary neurological diagnosis were considered in the analysis. We excluded patients receiving barbiturates and those without a DC mRS. The final analysis included 1,385 patients—1,200 for stroke and 185 for TBI—with more than 54,000 total pupillary measurements; mean age was 61.4 years; 706 (51%) patients were female and 1019 (73.6%) were Caucasian. Data were analyzed in Python (3.7.6) and MATLAB (R2020b, MathWorks). Nominal and ordinal data are reported as frequency (percent). Ratio and interval data are reported as mean (standard deviation). Difference between means tests using independent samples t-tests and confidence intervals (CIs) were calculated using statsmodels API. Receiver operating characteristic (ROC) analysis was conducted for evaluating the performance of the classifier and verifying the optimum threshold and the legitimacy of the cutoff (i.e. threshold differential) of 0.7 for NPI differential.

### Results

Length of stay in the ICU and frequency of pupillometry varied for each patient. In the example as shown in FIG. 10 a patient was monitored for >10 days with 4-5 observations per day. Right and left NPI values varied but their difference stayed below the cut-off reference (0.7) until around hour 100 when a few NPI differentials occurred. Note that, in this case, neither left nor right NPI ever fell below the critical value of 3.0. This patient depicted in FIG. 10 was discharged with severe disability: DC mRS=5.

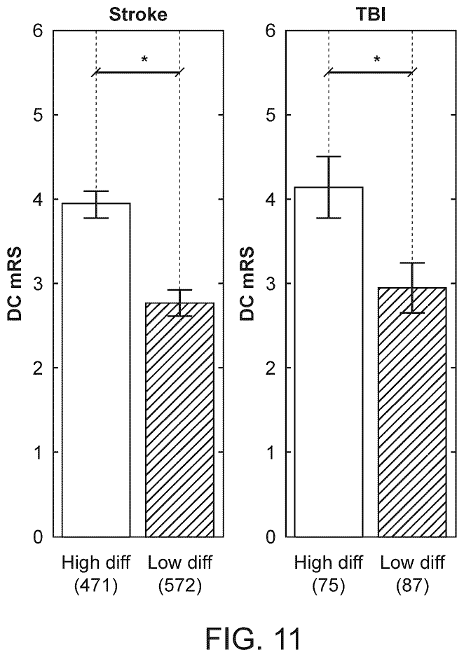

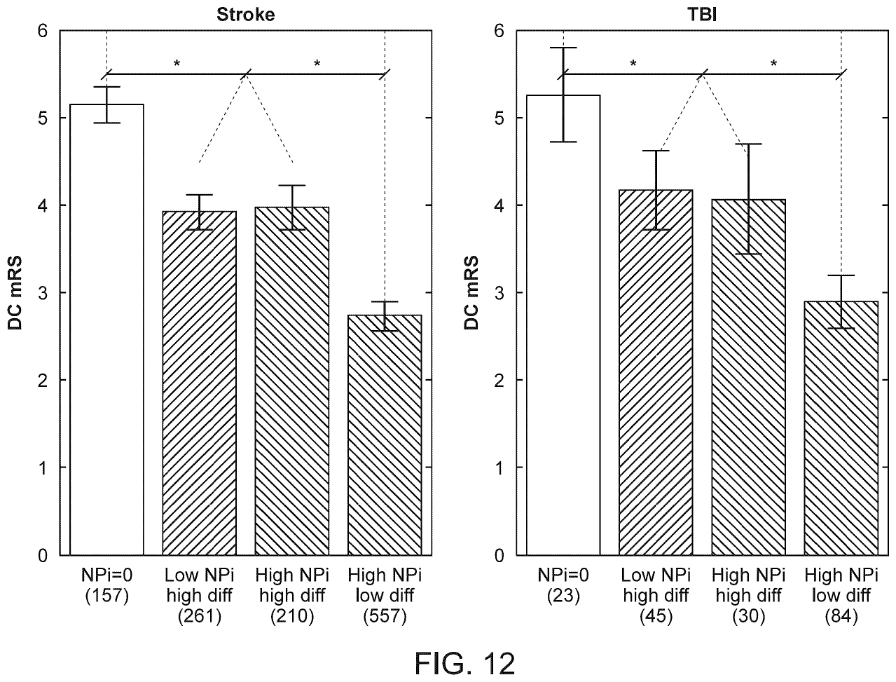

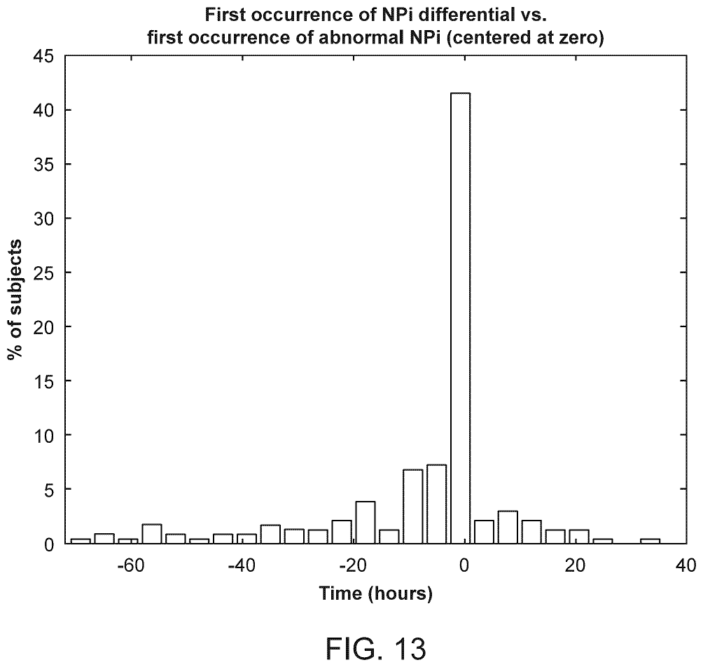

As shown in FIG. 11, patients were divided into two cohorts based on primary diagnosis, stroke (including subarachnoid hemorrhage¹¹, intracerebral hemorrhage¹², arterial ischemic stroke¹³, and aneurysm) and TBI (including subdural hematoma¹⁴), and NPI differences. Patients with at least one occurrence of an NPI differential (difference between the left and right NPI≥0.7) are designated as <high diff>. Patients with symmetric NPI's with differences always below 0.7 (<low diff>) are associated with a better outcome (lower DC mRS) in both cohorts (FIG. 11, see asterisks for P<0.001, mean DC mRS=2.8 vs. 3.9 for Stroke; 2.9 vs. 4.1 for TBI).

As shown in FIG. 12, all patients were then divided into four different groups according to their NPI differential and, this time, their NPI scores. One group corresponds to patients with at least one occurrence of a non-responsive pupil, <NPI=0>. The remaining are all patients whose pupils were always reactive. <low NPI high diff> are patients that had at least one occurrence of abnormal/low NPI (NPI<3.0) and at least one NPI differential (high diff); <high NPI high diff> are patients who had all normal/high (NPI>3.0) but at least one NPI differential (high diff); finally, <high NPI low diff> are patients with all normal NPI's and no NPI differentials. We omitted the <low NPI low diff> group (i.e. patients who had at least one abnormal/low NPI (NPI<3.0) but no NPI differentials) because of scarcity of data points—15 cases in stroke and only 3 in TBI. The group with NPI=0 was associated with the most severe DC mRS (mean DC mRS=5.2 for stroke and 5.3 for TBI). The following two groups, those having one or both abnormalities, were all significantly better than <NPI=0> (mean DC mRS=3.9 and 4.0 for stroke, 4.2 and 4.1 for TBI) but all equally poorer than the abnormality-free group <high NPI low diff> (mean DC mRS=2.7 for Stroke and 2.9 for TBI, FIG. 12, asterisks show P<0.001). Group <high NPI high diff> is particularly important as it represents cases with normal NPI's but with at least one NPI differential. This group exhibits the same poorer outcome as the group with both types of abnormality, <low NPI high diff> which further emphasizes the importance of the NPI differential even when the NPI is normal.

NPI differentials are not a rare phenomenon: in only 30% of all the cases in the two cohorts combined, they occurred only once and the average rate is approximately 8 times per patient. Our data are quite heterogeneous in terms of length of stay in the ICU and frequency of pupillary assessment and, thus, a rigorous analysis of the pattern or rate of incidence is not possible. Over the course of their stay in the ICU, nearly half of stroke and TBI patients had an NPI differential: stroke=45.11%, TBI=46.30%. This compares to the lower incidence of an abnormal NPI (among non-NPI=0 patients), stroke=26.48% and TBI=29.63%. If we use the first occurrence of an abnormal NPI (NPI<3.0) as a reference, say at time zero (Figure D) and we plot on the same time axis and for all subjects in the <low NPI high diff> group the time of the first occurrence of an NPI differential relative to that first abnormal NPI, we do find that i) the two abnormalities (NPI and differential) often occur exactly at the same time, i.e. they are part of the same pupillary assessment (see peak of histogram at time zero, Figure D) and, ii) the histogram is slightly skewed towards the left/negative end of the axis indicating an anticipatory effect of the NPI differential.

An ROC, developed to include all patients with DC mRS dichotomized as good (mRS 0-2) or poor (mRS 3-6) [36, 37] demonstrated an AUC=0.71 (P<0.001), with an optimal cutoff of >0.7.

### Discussion

The time waveform of the PLR is described by a number of variables representing the magnitude, velocity and latency of the reflex. NPI integrates all these variables into a multidimensional model based on a database of measurements collected on normal healthy individuals in different conditions^{1,2,15}. An NPI score ≥3.0 means that the pupil reactivity falls within the boundaries of the normative range (pupillary reaction to light is “brisk” or “normal”). An NPI score <3.0 denotes an abnormal pupillary light reflex which is outside the normal distribution (weaker than a normal response, or “sluggish”). Nonresponsive pupils are reported with an NPI=0. The same logic applies to the difference between the left and right NPI; values ≥0.7 are outside the variation observed in normal patients and are labelled “NPI differentials.” In healthy individuals, the NPI of both eyes should always be >3.0 and symmetric (<0.7 difference).

Abnormal values of the NPI index (NPI<3.0 or NPI=0) have been associated to worse outcomes in many different studies and applications^{1,4,16,17}. We unexpectedly found that NPI differentials have the same clinical implications as it relates to the DC mRS; they occur in patients with or without abnormal NPIs and are associated with a more severe DC mRS when compared to the abnormality-free group <high NPI low diff> (Figure C).

### Conclusion

Unexpectedly, the presence of an NPI differential exceeding a threshold differential is associated with a higher mRS at discharge, indicating a higher level of patient disability. More surprisingly, this association is consistent even in those circumstances when both NPI values are always normal (>3.0). Therefore, the NPI differential may be a prognostic indicator that clinicians should consider in decision making when managing patients with neurological injury.

### Example 2

A differential is a pair of measurements from the left and right eyes where the difference in their NPI values is equal to or greater than 0.7; these are referred to as NPI_differential, and in the examples in the graphs below, they are indicated with a horizontal gray bar. NPI differential persistence is a sequence of measurements where the difference between the left and right eye, although never equal to or greater than 0.7, consistently has the same sign. The graphs below are data taken from three different hospitalized patients with stroke, TBI, acute brain injury or other neurological impairment. In the first example below, the circle in the top panel shows a sequence of seven measurements where the right NPI was consistently smaller than the left NPI. In the second graph, there are four measurements with the same characteristic, and in the third graph, there are three. All these subjects demonstrate a persistence of the sign—it could be the right pupil being weaker (as in this examples) or the left pupil—the key condition is that as long as the direction or sign of the difference remains the same over two or more consecutive measurements, that is classified as persistency or NPI differential persistence. The data in the bar plot described above relating to NPI differential persistence demonstrates that NPI differential persistence is correlated with poor outcomes and it does not occur in normal healthy conditions.

While the invention is susceptible to various modifications and alternative forms, specific examples thereof have been shown by way of example in the drawings and are herein described in detail. It should be understood, however, that the invention is not to be limited to the particular forms or methods disclosed, but to the contrary, the invention is to cover all modifications, equivalents, and alternatives falling within the spirit and scope of the appended claims.

### BIBLIOGRAPHY

1. Riker, R.R., et al., Neurological Pupil Index and Pupillary Light Reflex by Pupillometry Predict Outcome Early After Cardiac Arrest. Neurocrit Care, 2020. 32(1): p. 152-161.
2. Chen, J.W., et al., Pupillary reactivity as an early indicator of increased intracranial pressure: The introduction of the Neurological Pupil index. Surg Neurol Int., 2011. 2: p. 82.
3. Olson, D.M. and M. Fishel, The Use of Automated Pupillometry in Critical Care. Crit Care Nurs Clin North Am, 2016. 28(1): p. 101-7.
4. Oddo, M., et al., Quantitative versus standard pupillary light reflex for early prognostication in comatose cardiac arrest patients: an international prospective multicenter double-blinded study. Intensive Care Med, 2018. 44(12): p. 2102-2111.
5. Kim, T.J., et al., Neurological Pupil Index as an Indicator of Neurological Worsening in Large Hemispheric Strokes. Neurocrit Care, 2020. 33(2): p. 575-581.
6. Lussier, B., et al., Distributions and Normal Ranges for Automated Pupillometer Values in Neurocritical Care Patients. Journal of Neuroscience Nursing, 2019. 51(6): p. 335-340.
7. Zhao, W., et al., Inter-device reliability of the NPI-100 pupillometer. Journal of Clinical Neuroscience., 2016. ePub.
8. Chen, J.W., et al., Infrared pupillometry, the Neurological Pupil index and unilateral pupillary dilation after traumatic brain injury: implications for treatment paradigms. Springerplus, 2014. 3: p. 548.
9. Olson, D.M., et al., Establishing Normative Data for Pupillometer Assessment in Neuroscience Intensive Care: The “END-PANIC” Registry. J Neurosci Nurs, 2017. 49(4): p. 251-254.
10. Natzeder, S., et al., Portable Infrared Pupillometer in Patients With Subarachnoid Hemorrhage: Prognostic Value and Circadian Rhythm of the Neurological Pupil Index (NPI). J Neurosurg Anesthesiol, 2018.
11. Adachi, H., et al., Clinical significance of pulse rate rise during sleep as a screening marker for the assessment of sleep fragmentation in sleep-disordered breathing. Sleep Med, 2003. 4(6): p. 537-42.
12. Priorities for clinical research in intracerebral hemorrhage: report from a National Institute of Neurological Disorders and Stroke workshop. Stroke, 2005. 36(3): p. e23-41.
13. 2005 American Heart Association (AHA) guidelines for cardiopulmonary resuscitation (CPR) and emergency cardiovascular care (ECC) of pediatric and neonatal patients: pediatric basic life support. Pediatrics, 2006. 117(5): p. e989-1004.
14. Al-Mufti, F. and S.A. Mayer, Neurocritical Care of Acute Subdural Hemorrhage. Neurosurg Clin N Am, 2017. 28(2): p. 267-278.
15. Al-Obaidi, S.Z., et al., Impact of Increased Intracranial Pressure on Pupillometry: A Replication Study. Crit Care Explor, 2019. 1(10): p. e0054.
16. Miroz, J.P., et al., Neurological Pupil index for Early Prognostication After Venoarterial Extracorporeal Membrane Oxygenation. Chest, 2020. 157(5): p. 1167-1174.
17. Ortega-Perez, S., et al., Pupillary Light Reflex Variability as a Predictor of Clinical Outcomes in Subarachnoid Hemorrhage. J Neurosci Nurs, 2019. 51(4): p. 171-175.

We claim:

1. A method of assessing a health condition of a mammalian subject comprising:
using a pupilometer to take three or more sequential measurements over a period of time of the left eye and the right eye of the subject and determining the NPI of each eye at each measurement, wherein the subject is hospitalized as a result of stroke, TBI, acute brain injury or other neurological impairment;
displaying on a display of the pupilometer a comparison between the NPI of the left and the NPI of right eye at each of the three or more sequential measurements;
determining whether the subject exhibits NPI differential persistence; and
assessing the subject with having an increased risk of a bad neurological or health outcome if the patient exhibits NPI differential persistence.
